(19) United States
(12) Patent Application Publication
(10) Pub. No.: US 2008/0195181 A1
(43) Pub. Date: Aug. 14, 2008

(54) Micro-Dermal Tone Skin Stimulator

(75) Inventor: Carol Cole, Encinitas, CA (US)

Correspondence Address: KNOBBE MARTENS OLSON & BEAR LLP, 2040 MAIN STREET, FOURTEENTH FLOOR, IRVINE, CA, 92614, US

(73) Assignee: CAROL COLE COMPANY, Encinitas, CA (US)

(21) Appl. No.: 11/911,321

(22) PCT Filed: Apr. 28, 2006
(86) PCT No.: PCT/US2006/016409

§ 371 Date: Oct. 26, 2007

Related U.S. Application Data
(60) Provisional application No. 60/675,517, filed on Apr. 28, 2005.

(51) Int. Cl. A61N 1/36

(52) U.S. Cl. 607/74

(57) ABSTRACT

A micro dermal tone skin stimulator system (2) that uses sale, painless, low-level electrical impulses to “reprogram” muscles to assure nearly original muscle shape and to support skin tissues with healthier blood circulation and more youthful firmness. The system comprises a portable hand-held unit with fully-integrated components, including a housing (22), battery pack, an internal circuit board, and an integrated pair of spherical toning probes (24A & 24B) for delivering stimulation to the skin. The circuit board generates a positive low-voltage square wave with 60 ms pulse duration for five cycles, then a negative square wave for five cycles, which repeats, delivering an adjustable 0 to 400 micro-amps output (adjustable by potentiometer). Delivering electrical impulses through the spherical probes (24A & 24B) using a gliding technique rehabilitates the muscles, improves circulation, texture and tone almost immediately, and continued use provides lasting clarity and restores freshness to one's appearance.

### BACKGROUND OF THE INVENTION

1. Field of the Invention

The present invention relates to skin care devices and, more particularly, to an electric facial stimulator.

2. Description of the Background

Electric stimulators have long been used for electrically stimulating muscles, skin, nerves, and hair for the purpose of improving circulation and general rehabilitation. In Asian cultures there is thought to be a synergistic effect when a stimulator of proper intensity and frequency is applied to specific areas.

Thomas W. Wing. D.C., N.D., LAC., a fifth generation Chinese Doctor is credited with introducing a microcurrent instrument in about 1974. By the 1980s he was using an electrical acupuncture device for the treatment of muscles to lengthen, shorten and strengthen. Dr. Wing's device was approved by the FDA as a muscle stimulator. However, it began to find a home in the Cosmetology industry for cellular regeneration (rejuvenation), facial toning and wrinkle reduction. It was found that microcurrent produces proliferation of cells by fibroblasts' secretion of proteins such as collagen. Additionally, it works on the nerves and muscle cells to increase tonicity of the fine facial muscles. Research has shown that the results of the microcurrent are not only dramatic but long lasting. Microcurrent heals and regenerates tissue; it is a corrective and preventive anti-aging treatment unlike other relative systems such as muscle stimulators whose temporary effect acts as a band-aid in the aging process.

Unfortunately, existing microcurrent systems were geared toward Electro-Muscle Stimulation (E,M.S.) which is a medical procedure utilizing a very strong current to force repeated muscular contraction and expansion. Moreover, these existing commercial electric stimulators require the application of conduction gel, patches, and a plurality of electrical connections, through which they generate low-frequency current pulses that are applied to the body. Typically, two conductive patches are adhered to the body in a manner very similar to ultrasound. Unfortunately, the use of such devices has heretofore been relatively inconvenient, and the results relatively disappointing.

Lasting skin beauty can only be achieved through overall health and by actually affecting the way the cells of the body function. Due to chemical, physical, and psychological stress, and the process of aging, cellular functions break down over time. All cells must constantly move new nourishment in, and the waste products of cell metabolism out. This process occurs through the cell membrane, and a delicate electrical balance is maintained. Changes in this electrical balance move material in and out of the cell. When the balance is upset, the cellular machinery does not function properly, and aging and degeneration occur. Moreover, suppleness and elasticity of the skin are affected by the quality of the collagen present in the cells. One effect of aging is gradual damage to the DNA and RNA molecules, causing deterioration of the collagen. The end result is that skin is no longer supple and taut as with youthful skin, but rather takes on a dull, sagging, and wrinkled appearance.

By stimulating the skin cells in a particular way, a user is capable of reducing wrinkles and sagging of the skin. It is therefore desirable to provide an electric stimulator to provide users with a convenient tool to delay aging of the skin and promote skin rejuvenation by accelerating collagen, elastin, and connective tissue production, and aid in the regrowth by stimulating mitotic activity and hemetic flow. It would, therefore, be greatly advantageous to provide a micro-dermal tone skin stimulator for stimulating the cells of one's facial skin and underlying tissues for repair, especially in a compact user-friendly footprint for home use.

### SUMMARY OF THE INVENTION

It is, therefore, the primary object of the present invention to provide a micro-dermal tone skin stimulator that stimulates the cells of one's facial skin and underlying tissues in such as way that allows the skin to repair itself and become healthier from the inside out by reestablishing the delicate electrical balance of the cells.

It is another object to provide a micro-dermal tone skin stimulator in a compact user-friendly handheld footprint for home use.

It is another object to provide a micro-dermal tone skin stimulator that delivers a positive low-voltage square wave with 50% duty cycle and 60 ms pulse duration for approximately five cycles, then a negative square wave with 50% duty cycle for five cycles, which repeats, the amplitude being adjustable within a range of from 0 to 250 mV (0 to 500 micro-amps), and the output pulses being adjustable within a range of from approximately 0.3 to 8 Hertz.

It is yet another object to provide a micro-dermal tone skin stimulator that delivers the foregoing output through a particular set of probes having spherical contact surfaces with a 0.5-0.6″ radius of curvature to ensure optimal contact with facial skin, maximum efficacy, and facilitate a particular gliding application technique.

These and other objects are accomplished by a micro dermal tone skin stimulator system that uses safe, painless, low-level electrical impulses to “reprogram” the muscles to assure nearly original muscle shape and to support skin tissues with healthier blood circulation and more youthful firmness. The gentle electrical impulses actually rehabilitate the muscles, improving circulation and improving texture, tone, and fine lines after only one treatment. Regular use of the micro dermal tone skin stimulator continues to provide clarity and restore freshness to one's appearance.

The micro dermal tone skin stimulator generally comprises a portable handheld unit with fully-integrated components designed for consumer home use. Rather than pads and conducting gel, the device employs two protruding side-by-side spherical toning probes of a particular size and intermediate spacing to deliver the electrical impulses, plus a particular gliding technique (made possible by the spherical electrodes) to maximize effectiveness. The components also include a hand-held base stimulator unit, battery pack, and an internal circuit board for generating low-voltage waveform-shaped current impulses. The circuit board generates a positive low-voltage square wave with 50% duty cycle and 60 ms pulse duration for five cycles, then a negative square wave with 50% duty cycle for five cycles, which repeats. Given an average 500 ohm load when the electrical impulses delivered through the probes to the facial skin, the output signal ranges from 0 to 250 mV (0 to 500 micro-amps), which is adjustable by on on-board potentiometer. The stimulation signal rehabilitates the muscles, improves circulation, texture and tone almost immediately, and continued use according to a weekly protocol provides lasting clarity and restores freshness to one's appearance.

Continued use according to the weekly protocol also disclosed herein provides lasting clarity and restores freshness to one's appearance.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

Other objects, features, and advantages of the present invention will become more apparent from the following detailed description of the preferred embodiments and certain modifications thereof when taken together with the accompanying drawings in which:

FIG. 1 is a rear perspective view of the micro dermal tone portable skin stimulator 20 according to one embodiment of the invention.

FIG. 2 is a front perspective view of the micro dermal tone portable skin stimulator 20 as in FIG. 1.

FIG. 3 is an exploded view of the housing 22 configuration used by the micro dermal tone portable skin stimulator 20 as in FIG. 1.

FIG. 4 is a block diagram of the electrical circuit resident on the circuit board in the portable skin stimulator 20 of FIG. 1.

FIG. 5 (5A & 5B) is a detailed schematic diagram of an exemplary circuit for the block diagram of FIG. 4.

FIG. 6 is a schematic drawing of an exemplary probe 24, inclusive of spherical probe tip 14 (with dimensions) for delivering the stimulating current.

### DETAILED DESCRIPTION OF THE PREFERRED EMBODIMENTS

The present invention is a micro dermal tone skin stimulator system for generating low-voltage waveform-shaped current impulses to the facial skin via specialized probe tips. The system is a portable unit with fully-integrated components designed for consumer home use.

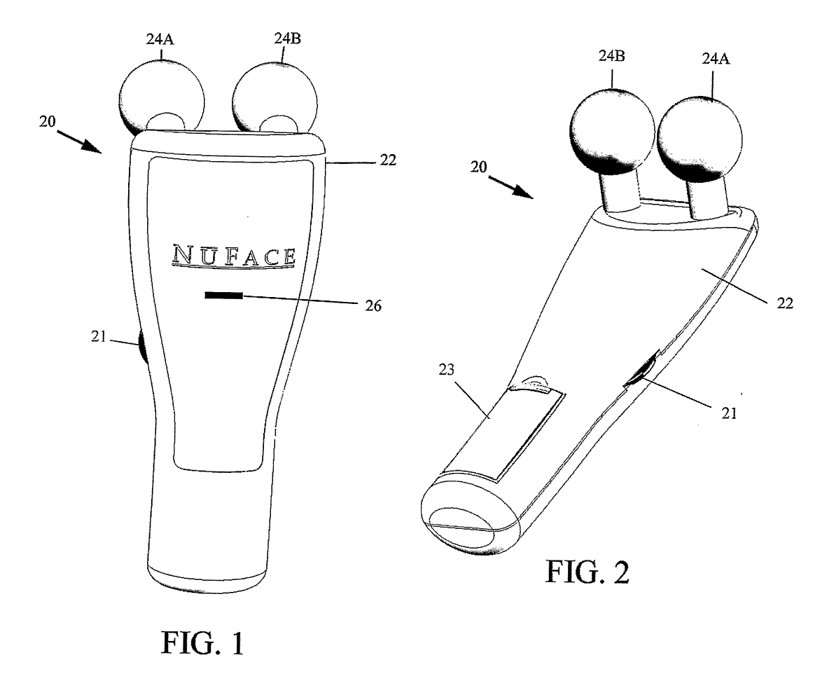

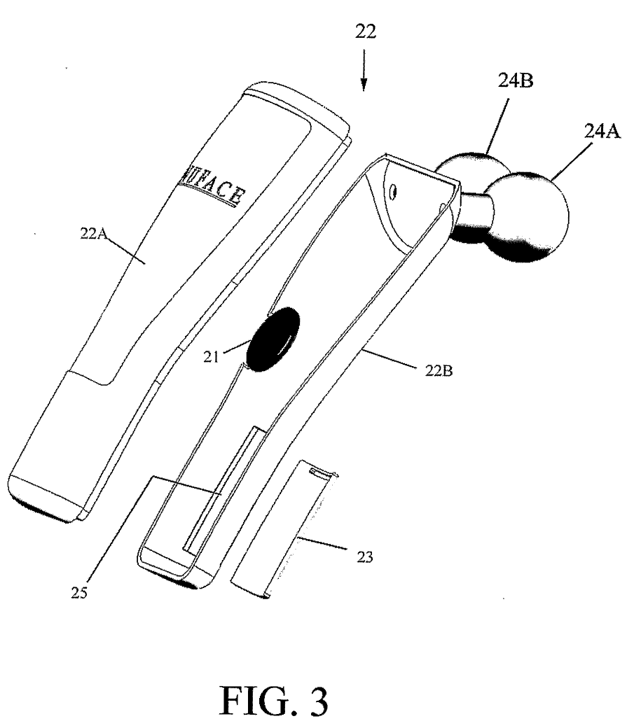

FIGS. 1 and 2 are a rear and front perspective view, respectively, of the micro dermal tone portable skin stimulator 20 which generally comprises a hand held base stimulator unit foored with a compact housing 22, a battery pack (internal) accessible through a removable hatch 23, and an internal circuit board (enclosed) for generating low-voltage waveform-shaped current impulses. By “hand held” it is meant that the entire device 20 may be easily grasped and manipulated for its intended purpose in one hand. In the illustrated embodiment the housing 22 is approximately 4.3″ long and 2.41 inches wide, contoured to fit the hand, and is molded of interfitting plastic halves.

An integrated pair of laterally-spaced toning probes 24A & 24B extend from the hand-held unit 22 at an operative end of the housing 22 for delivering stimulating current to the skin. The internal circuit board (to be described) generates a positive low-voltage square wave with 50% duty cycle and 60 ms pulse duration for approximately five cycles, then a negative square wave with 50% duty cycle for five cycles, which repeats. When tested using a 500 ohm load, the output signal delivered through the probes ranges from 0 to 250 mV (0 to 500 micro-amps), which is adjustable by on on-board potentiometer. The potentiometer on the circuit board (to be described) is mechanically coupled to a rotary adjustment dial 21 that protrudes through the housing for user-adjustment of the output signal amplitude. When the unit 20 is activated and the probes 24A & 24B are applied to the face in a gentle gliding manner, the stimulation signal rehabilitates the muscles, improves circulation, texture and tone almost immediately, and continued use according to a weekly protocol provides lasting clarity and restores freshness to one's appearance.

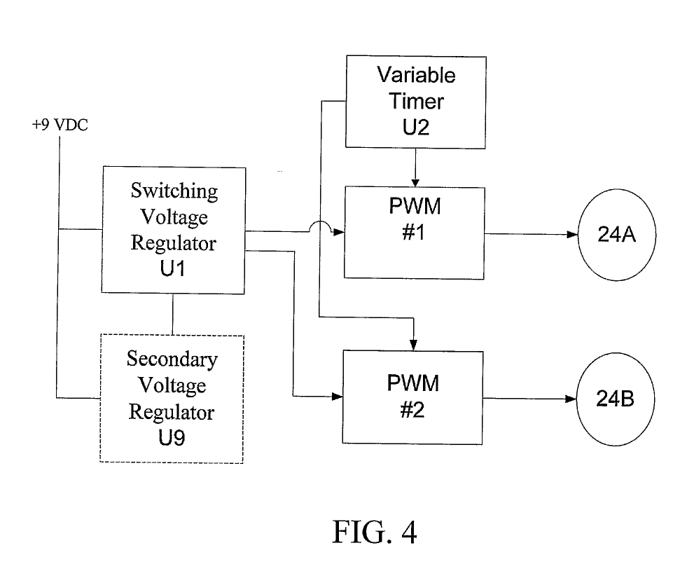

FIG. 3 is an exploded view of the housing 22 configuration of FIG. 1 which includes two interfitting halves 22A, 22B, and a battery compartment 25 with removable hatch 23. Base stimulator housing 22 encloses a standard 9 VDC battery in compartment 25 (not shown). Though not shown, the battery power source can be replaced or augmented by a standard plug-in AC/DC adapter that would attach to a panel-mount receptacle at the non-operative end of the enclosure (in which case the battery terminals would be disconnected when the plug-in AC/DC adapter is inserted). An LED power indicator light 26 is panel-mounted at some point (see, for example, FIG. 1) on the base stimulator housing 22 to indicate power. The housing 22 incorporates an internal circuit board (not shown in FIGS. 1-3) for generating low-voltage waveform-shaped current impulses, these impulses being delivered to the facial skin of a patient through the probes 24A & 24B which are extended distally from the end of the base stimulator unit 22 in a laterally-spaced relation. The entire unit 20 can be grasped and manipulated with one hand.

The probes 24A & 24b are equipped with specialized spherical probe tips for delivering the stimulating current to the skin. The probe tips (to be described) are mounted distally on short (0.5″) stems for ease of manipulation, and are internally connected to the circuit board in base stimulator unit 22. The particular radius of curvature of the distal application surface of these spherical toning probe tips as well as their lateral spacing facilitates proper delivery of the electrical impulses, and also facilitates a particular gliding technique (to be described) which maximizes effectiveness.

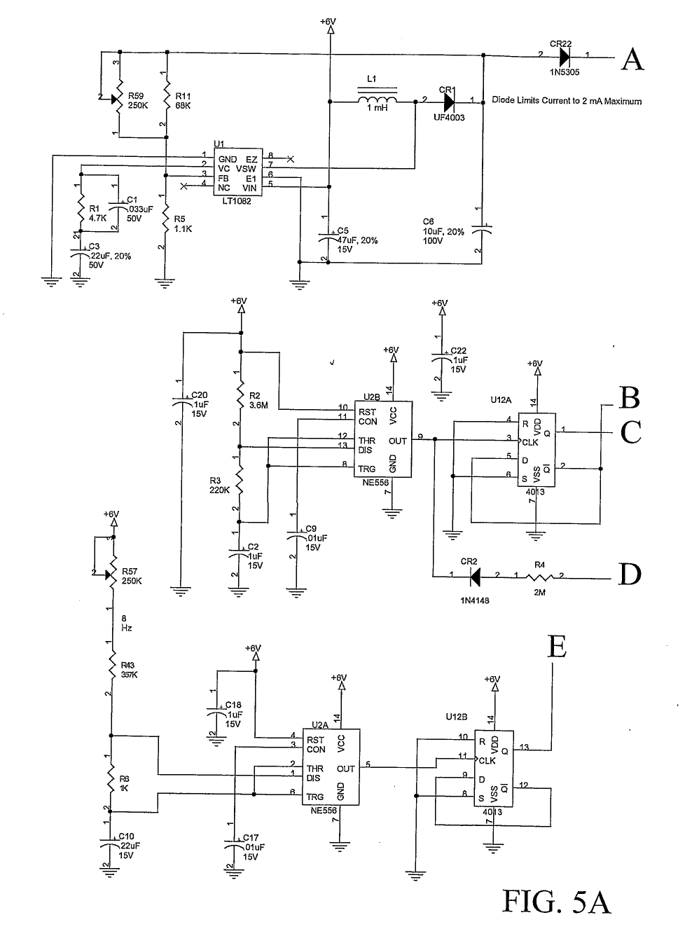

FIG. 4 is a block diagram of the electrical circuit resident on the circuit board in the portable skin stimulator 20 of FIG. 1, which is adapted for generating the low-voltage waveform-shaped current impulses. The basic circuit employs a Voltage Regulator U1 connected directly to the 9 VDC battery. Regulator U1 regulates the power output delivered to the probes 24A & 24B. For primarily battery-powered operation it is also wise to regulate the output voltage at a constant level despite draining battery power, and this may easily be accomplished with U9 which may be a linear or other suitable regulator. The regulated output drives a pair of pulse-width modulation (PWM) switches PWM #1 & PWM #2, which may be chopper circuits as described below. The timing and duration of the pulses output from PWM #1 & PWM #2 are controlled by a timing circuit U2, which is preferably variable. This allows user control over the output signal generated at probes 24A & 24B, which may be set within a range of from 0V to 250 mV (0 to 500 micro-amps), at a frequency between 0.3 to 8 Hz.

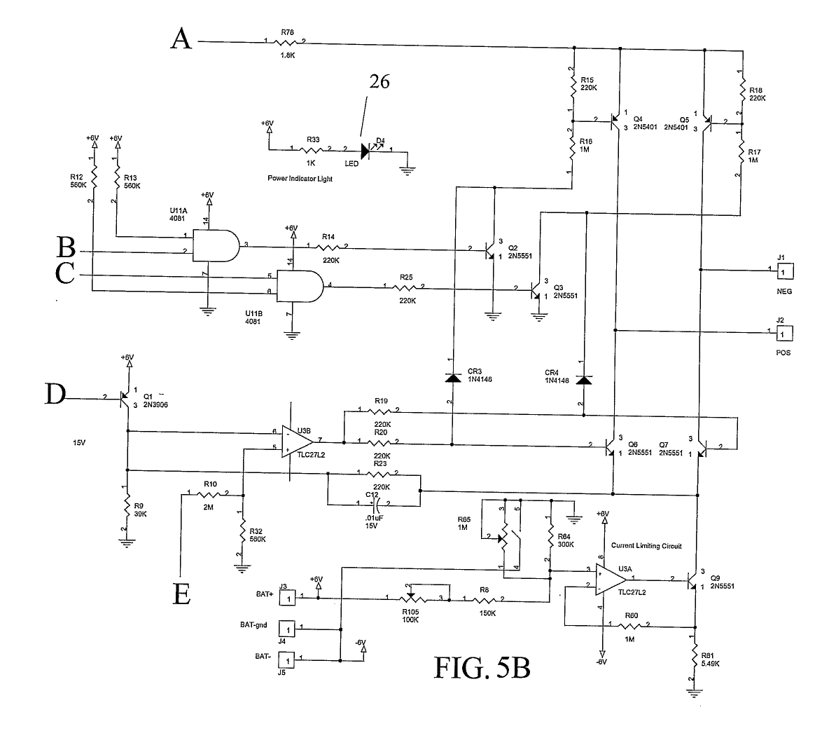

FIG. 5 (5A & 5B) is a schematic diagram of an exemplary electrical circuit for the block diagram of FIG. 4, which is resident on the circuit board in base stimulator unit 22 for generating the low-voltage waveform-shaped current impulses. This particular circuitry embodiment comprises a High Efficiency Switching Voltage Regulator U1 for regulating the power output delivered to the probes 24A & 24B (which are connected at J1 and J2 shown at right). Regulator U1 may be an LT1082 integrated circuit available from Linear Technology, Inc. or any number of other similar devices from multiple vendors. The Regulator U1 is setup in conventional boost switching configuration and takes any input voltage within the specified range of the device (3V to 75V) and outputs a voltage ranging from 25 to 100 or more volts. The regulator output voltage may be adjustable using a potentiometer or fixed by replacing the potentiometer with a resistor. All component values are preferably as shown in FIG. 5 (A & B). The regulator U1 draws power directly from the 9 VDC battery.

As seen in FIG. 4, for primarily battery-powered operation it may be desirable to include another voltage regulator (not shown in FIG. 5) to keep the output voltage constant despite draining battery power. This can easily be accomplished by augmenting the switching regulator U1 with a conventional linear regulator U9 (see FIG. 4), or alternatively a second high efficiency switching regulator, or a precision Schottky voltage reference. This secondary regulator controls the supply for timing electronics and maintains consistent product operation as the battery drains.

Also as stated above, the battery power may be augmented by a plug-in AC/DC adapter (6 VDC is suitable), in which case the battery power should be uncoupled when the 6V plug-in AC/DC adapter is inserted.

The +9 VDC power input to regulator U1 is protected by a current-limiting bridge circuit comprising inductor L1, Capacitors C5 and C6, and zener diode CR1. The 9V power bus is also connected to an LED power indicator light D4 (26 on FIG. 1) that is panel-mounted or side mounted on the base unit 6 to indicate power. The regulated output from regulator U1 is fed through a protective diode CR22 which limits output current to 2 mA and a pair of chopper circuits Q4, R15, R16 and Q5, R17, R18 which controllably pulse-width-modulate the regulated power output to form low-voltage waveform-shaped current impulses (positive and negative). When the transistors Q4 and Q5 are turned on, current is applied to the respective outputs J1 and J2 where it is delivered through the probes 24A & 24B. When turned off, no current flows. This is controlled to relative current impulses of reversing polarity on outputs J1 with respect to J2, e.g., positive and negative pulses. The timing and duration of these pulses is controlled for each output J1 and J2 by a respective timer circuit U2 (FIG. 4), which in the illustrated embodiment comprises an LM556 Dual timing circuit inclusive of timers U2A and U2B (FIG. 5). Similar devices such as TLC552 or TLC556 timers may be used. The LM556 is a highly stable controller capable of producing accurate time delays or oscillation, and is readily available from National Semiconductor; the TLS552 or TLS556 devices are pin-compatible devices produced by Texas Instruments. Motorola also produces similar devices. For the upper timer U2B, the timing is set in a known manner by external resistor/capacitors R2, R3, C20, C2 and C9. For the lower timer U2A, variable timing is set in a known manner by external resistor/capacitors R43, R6, C10 and variable resistor R57. The power to the LM556 (both U2A and U2B) is filtered by capacitors C17 and C18. Thus, U2B sets the timing for both positive and negative pulse-widths on J1 and J2, while U2A sets the frequency. Both timer outputs from U2A and U2B are fed through clock circuits U12A and U12B, the upper clock circuit U12A driving a pair of TTL logic AND gates U11A, U11B (each with one input tied to power) to form sharply delineated TTL pulse trains and to isolate the drive of the flip-flop U12 from the base of the NPN transistors Q2 and Q3. Other devices could be used to buffer the pulse train from U12A. These pulse trains drive transistors Q2 and Q3, which gate transistors Q4 and Q5 to apply the pulsed low voltage 6 VDC power to outputs J1 and J2, respectively, and on out through probes 24A & 24B. Lower timer output from U2A is also fed through a clock circuit U12B, and this feeds U3B, which is a Dual Precision Single Supply Power Operational Amplifier such as a Texas Instruments TLC27L2. This is compared to the output of timer U2B for selectively driving transistors Q6 and Q7, which control the frequency of the pulses on each output J1 and J2. It should now be apparent that the foregoing circuitry allows full PWM over the pulse-widths and frequency of both positive and negative pulses output to J1 and J2. This ensures sharp, fully-regulated low-voltage current impulses in accordance with factory pre-sets. The presets may be tested with a 500 ohm load. However, in this case the output voltage will need to be boosted to accommodate the variable impedance at the skin when the electrical impulses are delivered through the probes 24A & 24B to the facial skin. Again, the amplitude of the output signal is set within a range of from 0V to 250 mV (0 to 500 micro-amps), which is user-adjustable by on-board potentiometer R57 or could be fixed by replacing R57 with fixed resistor. Currently the output pulses range from approximately 0.3 to 8 Hertz.

The lower-right circuit formed by op-amp U3A is a conventional variable current regulator for running off 9 VDC battery power, from battery terminals BATT+, BATT−, and BATT Gnd as shown. Variable resistors R65 and R105 set the current regulation limits, while the maximum current is set by the value of R61 and can be modified for different applications of the product.

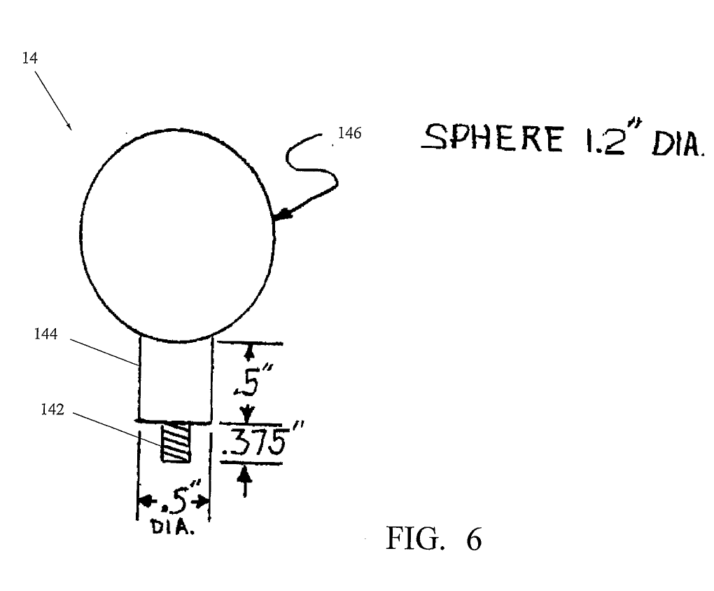

FIG. 6 is a schematic drawings of an exemplary probe 24, inclusive of spherical probe tip 14 (with dimensions) for delivering the stimulating current from J1 and J2 (FIG. 5) to the skin. The two probes 24A & 24B are adapted for screw-insertion into mating receptacles at the forefront of base stimulator unit 22, separated by approximately 0.5-1″, for ease of manipulation. In the preferred embodiment each probe 24 employs a fully spherical probe tip 14 with screw-threaded 0.375″ stem 142 for screw-insertion into the mating receptacle. One skilled in the art will understand that the two probes 24A & 24B may alternately be adapted for push-fit detent-lock insertion into the mating receptacles. The stem 142 of each probe tip 14 is integrally joined to a short cylindrical extension 144, which may be approximately 0.5″ long. The extension 144 is integrally joined to probe tip 14 which is a spherical electrode approximately 1.0-1.2″ diameter. The entire probe 24 inclusive of threaded stem 142, extension 144, and spherical electrode 14 may be integrally formed of chrome-plated aluminum, stainless steel or the like. One skilled in the art will understand that electrode need not be a full sphere to achieve the desired effect. A hemisphere or other partial sphere would also suffice, so long as the contact surface is spherical with a 0.5-0.6″ radius of curvature. The particular shape of spherical contact surface of probe tip 14, as well as its 0.5-0.6″ radius of curvature, plus the approximate 1.5-2.2″ intermediate spacing between the opposed distal contact points of the probe tips ensures optimal contact with facial skin, maximum efficacy, and facilitates the particular gliding technique described below.

In use, the micro dermal tone portable skin stimulator 20 is used according to an established protocol (below). Those who are pregnant, subject to seizures, or have a cardiac pacemaker, should not use the micro dermal tone portable skin stimulator 20. The protocol includes the following steps:

1. Begin with cleansing of the face.

2. Preferably, apply a conductive liquid or gel on the face and neck. This may be a coating of transmission gel, such as Conductor™ gel available from Chattanooga Group (a highly-conductive electrotherapy gel).

3. Adjust power level as desired to be comfortable and non-irritating.

4. Apply the micro dermal tone portable skin stimulator 20 beginning outside the center of the neck, pressing gently and gliding toward the back of the neck, taking steps up to under jaw line. Repeat this step 3 times. The spherical probes 24 described above are much more adept at this gliding motion than conventional sharp-tipped or drumstick-type probes as used for muscle stimulation.

5. Apply the micro dermal tone portable skin stimulator 20 outside the nasolabial folds, gliding towards the ear up the cheek bone to the top of the cheek bone. Repeat this step 3 times.

6. Apply the micro dermal tone portable skin stimulator 20 at the top of the brow, gliding up towards the hairline. Continue across the forehead until the end of the brow. Repeat this step 3 times.

7. Repeat the entire foregoing protocol 3-5 times a week.

It is important to note that there are areas to be avoided. It is improper to position the conducting applicator outside the mouth and eye areas. These entail circular muscles that must be avoided. Also avoid the midline (vertical center) of the neck. The foregoing protocol will rehabilitate the muscles, improve circulation, texture and tone. While this is apparent almost immediately after first use, the protocol of continued use provides lasting clarity and restores freshness to one's appearance.

The above-described micro dermal tone skin stimulator system 2 has been found to be both safe and effective during Botox™ treatments. Botox™ paralyzes the muscles and deadens the nerves causing muscles to atrophy, while diminishing blood supply in the skin. The micro dermal tone stimulator system 2 stimulates the muscles and nerves to maintain a healthier state, as well as stimulating blood flow. The micro dermal tone skin stimulator system 2 has also been found to be both safe and effective before or after Plastic Surgery. It rehabilitates the muscles to a firmer, normal state, thereby establishing a strong foundation and enhancing the surgeon's efficacy. After surgery, the system 2 reduces facial swelling, sustains the benefits of the procedure, and maintains long lasting results.

In normal use the above-described system 2 works by gently stimulating skin cells and the underlying muscles below the epidermis. Excessive fluids are removed and lymphatic drainage is increased, leading to decreased tissue swelling or “puffiness”. The device 2 will increase cellular blood flow, leading to increased cell turnover, leading to increased amounts of collagen, elastin, and connective tissue. This assists with skin rejuvenation, delays aging of the skin, and reduces the appearance of facial wrinkling.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

### INDUSTRIAL APPLICABILITY

There is demand within the Cosmetology industry for microcurrent devices for cellular regeneration (rejuvenation), facial toning and wrinkle reduction. Microcurrent produces proliferation of cells by fibroblasts' secretion of proteins such as collagen. Additionally, it works on the nerves and muscle cells to increase tonicity of the fine facial muscles. Existing microcurrent systems are medical grade devices that utilize a very strong current to force repeated muscular contraction and expansion. There are some that are designed for facial skin stimulation but they are large and expensive commercial electric stimulators for use by cosmetologists with their patients. They also use unwieldy conductive patches adhered to the body in a manner very similar to ultrasound. The use of such devices has heretofore been relatively inconvenient inasmuch as they require an appointment with a Cosmetologist, and the results relatively disappointing. Consequently, there is a significant commercial demand for a consumer-oriented microcurrent dermal tone stimulator for consumer home-use. The present invention is a micro-dermal tone skin stimulator in a compact user-friendly footprint for home use that effectively stimulates the cells of one's facial skin and underlying tissues for repair.

1. A portable system for electrical treatment of facial skin comprising:
a housing adapted to be grasped and manipulated by one hand;
a circuit board seated in said housing;
a battery for powering said circuit board; and
a pair of probes connected to said circuit board and protruding from said housing in a laterally-spaced relation and terminating at conductive probe tips;
said circuit board being adapted to deliver an intermittent low-voltage current to facial skin via the spherical contact surface of said probe tips at pre-determined pulse-widths, frequency and amplitude to rehabilitate muscles, and improve circulation, texture and tone.

2. The portable system for electrical treatment of facial skin according to claim 1, wherein said internal circuit board generates an intermittent low-voltage current comprising a series of positive square waves with 50% duty cycle followed by a series of negative square waves with 50% duty cycle, repeating.

3. The portable system for electrical treatment of facial skin according to claim 2, wherein said series of positive square waves comprises approximately five positive pulses, and said series of negative square waves comprises approximately five negative pulses.

4. The portable system for electrical treatment of facial skin according to claim 3, wherein each of said positive pulses and negative pulses comprises approximately a 60 ms pulse duration.

5. The portable system for electrical treatment of facial skin according to claim 4, wherein a frequency of said positive pulses and negative pulses is within a range of from 0.3 to 8 Hertz.

6. The portable system for electrical treatment of facial skin according to claim 2, wherein said internal circuit board generates an intermittent low-voltage current within a range of from 0 to 400 micro-amps.

7. The portable system for electrical treatment of facial skin according to claim 6, wherein said internal circuit board comprises a potentiometer connected to an adjustment switch on said housing for allowing user adjustment within said range of from 0 to 400 micro-amps.

8. The portable system for electrical treatment of facial skin according to claim 1, further comprising a pair of threaded receptacles seated in said housing for screw-insertion of said probes.

9. The portable system for electrical treatment of facial skin according to claim 8, wherein each of said probes comprises a threaded stem with distal conductive probe tip.

10. The portable system for electrical treatment of facial skin according to claim 9, wherein each of said conductive probe tips comprises a spherical contact surface.

11. The portable system for electrical treatment of facial skin according to claim 10, wherein the spherical contact surface of each of said conductive probe tips comprises a radius of curvature within a range of from 0.5 to 0.6 inches.

12. The portable system for electrical treatment of facial skin according to claim 11, wherein the foremost points on the spherical contact surface of said conductive probe tips are laterally spaced within a range of from 1.5-2.2 inches.

13. A portable system for electrical treatment of facial skin comprising:
a housing adapted to be grasped and manipulated by one hand;
a circuit board seated in said housing;
a battery for powering said circuit board; and
a pair of probes connected to said circuit board and protruding from said housing in a laterally-spaced relation and terminating at conductive probe tips defined by a spherical contact surface with a radius of curvature within a range of from 0.5 to 0.6 inches;
said circuit board being adapted to deliver an intermittent low-voltage current to facial skin via the spherical contact surface of said probe tips at pre-determined pulse-widths, frequency and amplitude to rehabilitate muscles, and improve circulation, texture and tone.

14. The portable system for electrical treatment of facial skin according to claim 13, wherein said internal circuit board generates an intermittent low-voltage current comprising a series of positive square waves with 50% duty cycle followed by a series of negative square waves with 50% duty cycle, repeating.

15. The portable system for electrical treatment of facial skin according to claim 14, wherein said series of positive square waves comprises approximately five positive pulses, and said series of negative square waves comprises approximately five negative pulses.

16. The portable system for electrical treatment of facial skin according to claim 15, wherein each of said positive pulses and negative pulses comprises approximately a 60 ms pulse duration.

17. The portable system for electrical treatment of facial skin according to claim 16, wherein a frequency of said positive pulses and negative pulses is within a range of from 0.3 to 8 Hertz.

18. The portable system for electrical treatment of facial skin according to claim 17, wherein said internal circuit board generates an intermittent low-voltage current within a range of from 0 to 400 micro-amps.

19. The portable system for electrical treatment of facial skin according to claim 18, wherein said internal circuit board comprises a potentiometer connected to an adjustment switch on said housing for allowing user adjustment within said range of from 0 to 400 micro-amps.

20. A method for improving texture and tone of facial skin using a portable adjustable-micro-current system for electrical treatment with a pair of probes protruding from said system in a laterally-spaced relation and terminating at conductive probe tips, comprising the steps of:
adjusting an amplitude of said micro current for comfort;
applying the probes of said micro-current system outside the center of the neck, pressing gently and gliding toward the back of the neck, gliding up to under the jaw line;
applying the probes of said micro-current system outside the nasolabial folds, gliding towards the ear, up to the cheek bone;
applying the probes of said micro-current system at the top of the brow, gliding up towards the hairline, and continuing across the forehead until the end of the brow.
